SEQUENCE LISTING (19 sequences)

SEQ ID NO: 1 - protein, 9 aa, Artificial sequence
  Synthetic peptide
DEXEEXASC

SEQ ID NO: 2 - protein, 10 aa, Artificial sequence
  Synthetic peptide
DEMEECASHL

SEQ ID NO: 3 - DNA, 19 bp, Artificial sequence
  Synthetic oligonucleotide
gaaggtgaaggtcggagtc

SEQ ID NO: 4 - DNA, 20 bp, Artificial sequence
  Synthetic oligonucleotide
gaagatggtgatgggatttc

SEQ ID NO: 5 - DNA, 19 bp, Artificial sequence
  Synthetic oligonucleotide
tctgcggaaccggtgagta

SEQ ID NO: 6 - DNA, 19 bp, Artificial sequence
  Synthetic oligonucleotide
tcaggcagtaccacaaggc

SEQ ID NO: 7 - protein, 9 aa, Artificial sequence
  Synthetic peptide
KARVLAEAM

SEQ ID NO: 8 - protein, 8 aa, Artificial Sequence
  Synthetic peptide
RQANFLGK

SEQ ID NO: 9 - protein, 8 aa, Artificial Sequence
  Synthetic peptide
SQNYPIVQ

SEQ ID NO: 10 - protein, 12 aa, Artificial Sequence
  Synthetic peptide
RTNKKRSWPLNE

SEQ ID NO: 11 - protein, 12 aa, Artificial Sequence
  Synthetic peptide
EVKKQRAGVLWD

SEQ ID NO: 12 - protein, 12 aa, Artificial Sequence
  Synthetic peptide
FAAGRKSLTLNL

SEQ ID NO: 13 - protein, 12 aa, Artificial Sequence
  Synthetic peptide
TTNTRRGTGNIG

SEQ ID NO: 14 - protein, 12 aa, Artificial Sequence
  Synthetic peptide
KGASRRSWPLNE

SEQ ID NO: 15 - protein, 12 aa, Artificial Sequence
  Synthetic peptide
QVKTQRSGALWD

SEQ ID NO: 16 - protein, 6 aa, Artificial Sequence
  Synthetic peptide
TLFQGP

SEQ ID NO: 17 - protein, 16 aa, Artificial Sequence
  Synthetic peptide
DVMTAIFQGPIDMKNP

SEQ ID NO: 18 - protein, 14 aa, Artificial Sequence
  Synthetic peptide
EPDFHLQGPEDLAK

SEQ ID NO: 19 - protein, 8 aa, Artificial Sequence
  Synthetic peptide
SRGGGTCA

(19) United States
(12) Patent Application Publication
(10) Pub. No.: US 2010/0099079 A1
(43) Pub. Date: Apr. 22, 2010

(54) Non-dividing cell-based assay for high throughput antiviral compound screening

(76) Inventors: Susan L. Uprichard, Chicago, IL (US); Xuemei Yu, Chicago, IL (US); Bruno Sainz, JR., Chicago, IL (US)

Correspondence Address: LICATA & TYRRELL P.C., 66 E. MAIN STREET, MARLTON, NJ, 08053, US

(21) Appl. No.: 12/566,074

(22) Filed: Sep. 24, 2009

Related U.S. Application Data
(60) Provisional application No. 61/100,540, filed on Sep. 26, 2008.

(51) Int. Cl. C12Q 1/70

(52) U.S. Cl. 435/5

(57) ABSTRACT

The present invention features a cell-based assay that recapitulates all aspects of a viral lifecycle for use in identifying antiviral agents. The assay employs synchronized, non-dividing host cells and a fluorescence resonance energy transfer peptide substrate for monitoring endogenous viral protease activity, which is indicative of viral infection kinetics.

This application claims the benefit of priority of U.S. Provisional Application No. 61/100,540, filed Sep. 26, 2008, the content of which is incorporated herein by reference in its entirety.

This invention was made in the course of research sponsored by the National Institutes of Health (Grant No. R01-AI070827). The U.S. government has certain rights in this invention.

### BACKGROUND OF THE INVENTION

Hepatitis C virus (HCV) is an enveloped positive-strand RNA virus that infects and replicates in the liver of ˜170 million individuals worldwide. Although acute infection is typically asymptomatic, ˜80% of patients fail to clear the virus resulting in a chronic infection associated with significant liver disease, including cirrhosis and hepatocellular carcinoma (HCC) (Alter & Seeff (2000) Semin. Liver Dis. 20:17-35). As such, in the United States, HCV-related HCC accounts for over 50% of HCC cases and over 30% of liver transplants performed. With no vaccine available to protect against HCV infection and only a subset of chronically-infected patients responding to current treatment options (Ahmed & Keeffe (1999) J. Gastroenterol. Hepatol. 14 Suppl:S12-8), there is an immediate need for new effective HCV antivirals.

HCV is classified in the family Flaviviridae based on conservation of the viral RNA-Dependent RNA Polymerase (RDRP) and genome organization (Lindenbach & Rice (2005) Nature 436:933-8). The −9.6 kb RNA genome encodes a single open reading frame flanked by highly structured 5′ and 3′ untranslated regions (UTRs). The 5′ UTR contains an internal ribosome entry site (IRES) that is required for translation of a ˜3010 amino acid viral polyprotein, which is proteolytically cleaved into structural and non-structural (NS) proteins. The NS viral proteins assemble on cytoplasmic cellular membranes to form the viral RNA replication complex where negative strand RNA synthesis is believed to occur (Gosert, et al. (2003) J. Virol. 77:5487-92). The negative strand then provides the template for ˜10-fold amplification of positive strand genomic RNA, which can subsequently be used for additional translation, negative-strand synthesis, or packaging into progeny virus (Lindenbach & Rice (2005) supra).

Since its discovery as the causative agent of non-A non-B hepatitis (Choo, et al. (1989) Science 244:359-362), the viral lifecycle and host-virus interactions that determine infection outcome have been difficult to study. Nonetheless, significant advancements in the study of HCV have been made using surrogate systems (Beames, et al. (2001) Ilar J. 42:152-60), sub-genomic and full-length HCV replicons (Blight, et al. (2000) Science 290:1972-5; Blight, et al. (2003) J. Virol. 77:3181-90; Ikeda, et al. (2002) J. Virol. 76:2997-3006; Lohmann, et al. (1999) Science 285:110-3) and pseudotyped particles (HCVpp) (Bartosch, et al. (2003) J. Exp. Med. 197:633-42). While the HCV replicon and HCVpp systems were breakthroughs that overcame key experimental limitations, these systems only afford the study of viral replication and entry, respectively, and do not recapitulate the entire viral lifecycle. It was not until the genotype 2a HCV consensus clone (JFH-1) was shown to replicate in the Huh7 human hepatoma-derived cell line and produce infectious HCV in cell culture (HCVcc) (Lindenbach, et al. (2005) Science 309:623-6; Wakita, et al. (2005) Nat. Med. 11:791-6; Zhong, et al. (2005) Proc. Natl. Acad. Sci. USA 102:9294-99), that all aspects of the viral lifecycle were recapitulated.

Although numerous HCV replicon-based high-throughput screening (HTS) assays have been developed (Bourne, et al. (2005) Antiviral Res. 67:76-82; Dansako, et al. (2008) Virus Res. 137:72-9; Hao, et al. (2007) Antimicrob. Agents Chemother. 51:95-102; Huang, et al. (2008) Antimicrob. Agents Chemother. 52:1419-29; Kim, et al. (2007) Gastroenterology 132:311-20; Lee, et al. (2003) Anal. Biochem. 316:162-70; Lee, et al. (2005) Assay Drug Dev. Technol. 3:385-92; Mao, et al. (2003) World J. Gastroenterol. 9:2474-9; Mondal, et al. (2009) Antiviral Res. 82:82-8; O'Boyle, et al. (2005) Antimicrob. Agents Chemother. 49:1346-53; Zuck, et al. (2004) Anal. Biochem. 334:344-55; U.S. Pat. No. 7,195,885; and US Patent Application Nos. 20030215917 and 20050260568), the need to screen compounds that target all steps of the HCV lifecycle is warranted.

### SUMMARY OF THE INVENTION

The present invention features a method for identifying an antiviral agent. The method involves infecting a non-dividing host cell culture with an infectious virus that expresses a protease integral to the lifecycle of the virus; contacting said host cell culture with a test agent and a peptide substrate for said protease; incubating the host cell culture for a time sufficient to complete at least one lifecycle of the virus; and determining activity of the protease using the protease substrate, wherein a decrease of protease activity identifies the test agent as an antiviral agent. In one embodiment, the virus is a Retroviridea virus, a Flaviviridea virus such as a hepatic virus, a Picornaviridea virus, a Caliciviridea virus, a Togaviridea virus, or a Coronaviridea virus. In another embodiment, the host cell culture is infected at a multiplicity of infection of less than 0.1 focus forming units/cell. In further embodiments, the host cell culture is contacted with the test agent before or at the time of infection. In particular embodiments, the host cell culture is contacted with the test agent during the exponential phase of viral spread through the host cell culture. In a further embodiment, the lifecycle of the virus comprises host cell binding, entry, uncoating, translation, replication, assembly, maturation, egress and spread. According to other embodiments, the peptide substrate is labeled, e.g., with dyes capable of FRET fluorescence, wherein the FRET fluorescence is measured continuously, intermittently or at a specified time point. In other embodiments, the method is performed in a high-throughput manner, and the assay further includes the step of assessing the cytotoxicity of test agent.

### BRIEF DESCRIPTION OF THE DRAWINGS

FIG. 1 shows the quantitative identification of inhibitors that act throughout the HCV lifecycle. FIG. 1A, HTS experimental design. FIG. 1B, DMSO-Huh7 cells were infected with HCV at 0.05 FFU/cell and treated with: 2.5 μM CsA, 100 U/ml IFN-α, 100 U/ml IFN-β, 100 U/ml IFN-γ, 10 μM MA, and 18.5 μM NM107. Compounds were added 2 days post-infection and were replenished in fresh medium day 4 post-infection. Day 6 post-infection, triplicate cultures were assayed for HCV RNA levels by RT-qPCR and NS3 protein levels by FRET. Data are presented as a percentage of mock-treated. FIG. 1C, DMSO-Huh7 cells were infected with HCV at 0.05 FFU/cell and treated with HCV inhibitors that act at different stages of HCV infection: 50 μg/ml α-CD81, 100 μg/ml α-E2, 2.5 μM CsA, 250 U/ml IFN-β, 18.5 μM NM107, 200 μM Naringenin, and 500 μM NB-DNJ. Compounds were added at the time of infection and were replenished every 2 days over the 6 day assay. Day 6 post-infection, triplicate cultures were assayed for HCV RNA levels by RT-qPCR and NS3 protein levels by FRET. Data are presented as a percentage of mock-treated. FIG. 1D, DMSO-Huh7 cells were infected with HCV at 0.05 FFU/cell and treated post-infection on days 2 and 4 post-infection with HCV inhibitors that act at different stages of HCV infection: 50 μg/ml α-CD81, 100 μg/ml α-E2, 2.5 μM CsA, 250 U/ml IFN-β, 18.5 μM NM107, 200 μM Naringenin, and 500 μM NB-DNJ. Day 6 p.i., triplicate cultures were assayed for HCV RNA levels by RT-qPCR and NS3 protein levels by FRET. Data are presented as a percentage of mock-treated.

### DETAILED DESCRIPTION OF THE INVENTION

A simple, mix-and-measure, homogenous, cell-based viral infection assay has now been developed for HTS of antiviral agents. This assay makes use of synchronized, non-dividing host cells, which support more reproducible long-term viral infection and can be readily scaled to any format. Furthermore, instead of using exogenous or foreign enzymatic reporters to measure viral infection, the assay described herein uses endogenous protease activity as a virally-encoded “enzymatic reporter” of virus infection. This strategy is based on the observation herein that HCV NS3 protease activity parallels HCV infection kinetics and the ability of the viral NS3 protein to cleave internally quenched peptide substrates (Bianchi, et al. (1996) Anal. Biochem. 237:239-44; Kakiuchi, et al. (1999) J. Virol. Methods 80:77-84) allowing for quantitative measurement of protease activity by FRET. This stable cell-based method eliminates common problems associated with standard cell-based HTS, such as cell culture variability, poor reproducibility and low signal intensity. In addition, the use of non-dividing host cells allows for long-term viral infections thereby allowing the identification of inhibitors that act throughout the viral life cycle. The result is a high degree of accuracy with limited signal variation (i.e., Z′≧0.6), providing the basis for a robust HTS campaign for screening compound libraries and identifying novel antiviral agents for the prevention and/or treatment of viral infections.

Accordingly, the present invention features an assay for identifying an antiviral agent which involves infecting a non-dividing host cell culture with an infectious virus that expresses a protease integral to the lifecycle of the virus; contacting said host cell culture with a test agent and a peptide substrate for said protease; incubating the host cell culture for a time sufficient to complete at least one lifecycle of the virus and allow for spread; and measuring protease activity, wherein a decrease of protease activity identifies the test agent as an antiviral agent. Unlike replicon systems that specifically assay viral RNA replication, the infectious cell culture system of the invention recapitulates all aspects of the viral lifecycle, such as binding, entry, uncoating, translation, replication, assembly, maturation, egress and spread. This is a considerable advantage as it provides the opportunity to identify compounds that inhibit any step in the viral lifecycle.

A non-dividing host cell culture, as used herein, refers to a cell culture, the growth of which has been arrested. Exemplary viral host cells and methods for arresting growth are described herein and known in the art. In particular embodiments, the cell culture of the invention is synchronized. Cells can be synchronized by serum starvation before releasing them from this state, or by treating the cells with chemical inhibitors which arrest cells in distinct phases of the cycle. In so far as the host cell cultures of the instant assay are for use in identifying antiviral agents throughout the lifecycle of a virus, desirably the host cell cultures are capable of being infected by and allow for completion of at least one, two, three, four, or more complete viral lifecycles of a virus disclosed herein. While illustrative examples of host cell cultures and methods for obtaining synchronized, non-dividing host cell cultures are provided herein, any suitable host cell culture and method can be employed.

Advantageously, the growth arrested steady-state nature of the host cell cultures herein virtually eliminates the complications inherent to cell-based HTS assays, such as cell culture-related variability from well-to-well. In addition, the non-dividing host cell cultures allow for high reproducibility and robust infection over an extended period of time rather than only a few days making it feasible to adapt longer term infection strategies that allow for multiple rounds of viral replication and spread, a feature not afforded by conventional cell-based HTS assays.

By way of illustration, the use of the well-characterized, non-dividing Huh7 cell cultures (Choi, et al. (2009) Xenobiotica 39:205-17; Sainz, Jr. & Chisari (2006) J. Virol. 80:10253-7) also imparts several other advantages. Aside from the ease with which these ready-for-use cultures can be maintained and their inherent tolerability to the common compound library diluent DMSO, a more tangible benefit is that these cell cultures are resistant to many of the non-specific effects some compounds can have on the growth and viability of actively dividing cell cultures, which routinely result in false positive hits. While this does not eliminate the need to screen for compound cytotoxicity, the fact that the cultures are maintained in a quiescent non-proliferating state reduces the need for additional secondary screens to monitor compound-mediated effects on cell growth. Lastly, culturing Huh7 cells under these non-dividing conditions also results in enhanced Huh7 cell differentiation with the up-regulation of liver-specific gene expression (Choi, et al. (2009) supra; Sainz, Jr. & Chisari (2006) supra) and hepatocyte-specific Phase I and Phase II drug metabolism functions (Choi, et al. (2009) supra). The use of metabolically active cells would prove highly beneficial when screening pro-drug compounds, which need to be metabolized to an active form in order to exert their potential antiviral affect. Thus, in particular embodiments, the present invention embraces the use of Huh7 cells, also known as Huh7/scr cells (Gastaminza, et al. (2006) J. Virol. 80:11074-81; Zhong, et al. (2006) J. Virol. 80:11082-93). These cells are known in the art and can be obtained from sources such as the Health Science Research Resources Bank (HSRRB, Osaka, Japan) under JCRB No. 0403.

According to particular embodiments, the instant assay is carried out in the identification of an antiviral agent targeting a Retroviridea virus (e.g., human immunodeficiency virus), a Flaviviridea virus (e.g., hepatic viruses or dengue virus), a Picornaviridea virus (e.g., rhinovirus), a Caliciviridea virus (e.g., Norwalk virus), a Togaviridea virus (e.g., rubella virus), or a Coronaviridea virus (e.g., SARS coronavirus) or an enterovirus (e.g., Poliovirus, coxsackie virus and echoviruses). As is known in the art and described herein, viruses in these families express proteases that are integral or essential for completion of the viral lifecycle. Advantageously, the instant assay employs the endogenous viral protease activity as a “virally encoded reporter” for monitoring the lifecycle of the virus. In this respect, when the host cell culture is incubated for a time sufficient to complete lifecycle of the virus (e.g., as determined by known conditions or by determining levels of viral RNA), an agent identified as an antiviral agent will decrease protease activity as the virus being assayed will be unable to complete one or more of host cell binding, entry, uncoating, translation, replication, assembly, maturation egress and/or spread and thus fail to amplify. In so far as viruses can encode other enzymes, it is contemplated that the instant assay could be modified to use any other endogenous enzyme as a reporter. Likewise, it is contemplated that an exogenous reporter could also be used.

In particular embodiments, the instant assay is used in the identification of agents useful in the prevention and/or treatment of hepatic viruses. The term “hepatic virus” refers to a virus that can cause viral hepatitis. Viruses that can cause viral hepatitis include hepatitis A, hepatitis B, hepatitis C, hepatitis D, and hepatitis E. In addition, non-ABCDE cases of viral hepatitis have also been reported (see, for example, Rochling, et al. (1997) Hepatology 25:478-483). Within each type of viral hepatitis, several subgroupings have been identified.

Hepatitis C, for example, has at least six distinct genotypes (1, 2, 3, 4, 5, and 6), which have been further categorized into subtypes (e.g., 1a, 1b, 2a, 2b, 2c, 3a, 4a) (Simmonds (2004) J. Gen. Virol. 85:3173-3188). In particular embodiments of the invention, the hepatic virus is hepatitis C virus (HCV).

In so far as viral protease activity parallels infection kinetics, a low multiplicity of infection (MOI) infection assay approach can be used, wherein all aspects of the viral lifecycle including binding, entry, uncoating, translation, replication, assembly, maturation, egress and/or spread can be targeted. Accordingly, in particular embodiments of the invention, the host cell culture is infected at a MOI of less than 0.1 focus forming units (FFU)/cell, or more desirably less than 0.05 FFU/cell.

To monitor endogenous viral protease activity, the present assay employs a peptide substrate, the cleavage of which by its cognate viral protease is detectable. Viral protease activity, based upon cleavage of the peptide substrate, can be determined using any conventional assay. Desirably, the assay employed uses a labeled peptide substrate. For example, the assay can be based on a GAL4 inactivation assay (Lawler & Snyder (1999) Anal. Biochem. 269:133-138), wherein the protease substrate is labeled with the DNA binding and transactivating domains of GAL4 such that, upon the proteolytic cleavage of the peptide substrate, GAL4 dissociates and expression of luciferase is decreased. In another suitable assay, the protease substrate is labeled with enhanced green fluorescent protein (EGFP) and secreted alkaline phosphatase (SEAP), wherein secretion of SEAP into the culture medium is dependent upon the cleavage of the peptide substrate by the viral protease (Lee, et al. (2003) Anal. Biochem. 316:162-170).

In particular embodiments, the assay is based on a FRET approach. The basis of FRET is to bring a fluorescing dye close enough to a dye that prevents fluorescence (quencher) by coupling the dyes to a peptide that is a substrate for the protease being tested. Once the protease has severed the peptide substrate, the fluorescing dye can now separate far enough away from the quencher to produce a detectable signal. It is contemplated that any suitable combination of dyes can be employed. However, in particular embodiments, QXL™ dyes (Anaspec) are employed as they are individually optimized to pair with conventional fluorescent dyes such as fluoresceins and rhodamines. The QXL™ series of nonfluorescent dyes cover the full visible spectrum with high efficiency. QXL™ 520 has an absorption maximum matching the emission of FAM, whereas QXL™ 570 is a suitable quencher for TAMRA, and QXL™ 670 and 680 are the most effective quenchers for Cy₅ and Cy₅-like fluorophores. In general, the mechanics for the quenching can vary depending on the dye and quencher combination, but the concept at the technological level remains the same. Once the peptide substrate is cleaved, the fluorescent dye can separate far enough away from the quencher for the fluorescent emission to escape and be detected.

Peptide substrates for endogenous viral proteases are known in the art, and illustrative examples are provided herein. Additional peptide substrates for the viral proteases discussed herein are available from the MEROPS database located on the world-wide web (see Rawlings, et al. (2002) Nucl. Acids Res. 30:343-346). It is contemplated that any conventional methodology can be used to conjugate or attach labels to the ends of the peptide substrate. For example, wherein the peptides substrate is fused between two proteins (e.g., GAL4 DNA binding and transactivating domains, or EGFP and SEAP), the peptide substrate can be expressed in-frame as a fusion protein according to conventional recombinant protein technology. By way of further illustration, a thiol-reactive dye (e.g., a maleimide derivative of a dye) can be conjugated to a peptide substrate containing a sulfhydryl group (e.g., a cysteine amino acid residue). An exemplary labeled HCV NS3 protease substrate is Ac-Asp-Glu-Dap-Glu-Glu-Abu-ψ-[COO]-Ala-Ser-Cys-NH₂ (SEQ ID NO:1), wherein QXL™ 520 is conjugated to Dap and 5-FAMsp is conjugated to Cys. The sequence of this FRET peptide is derived from the sequence Asp-Glu-Met-Glu-Glu-Cys-Ala-Ser-His-Leu (SEQ ID NO:2), which is the natural cleavage site of NS4A/NS4B. The cysteine on the natural cleavage site is replaced with aminobutyric acid (Abu) and the scissile amide bond with an ester bond.

The manner in which the host cell is contacted with the protease substrate will be dependent upon the approach used to determine protease activity (e.g., GAL4 dissociation, SEAP secretion or FRET). For example, when it is desirable to determine the protease substrate in an intact cell (e.g., in the GAL4 dissociation or SEAP secretion assays), the protease substrate can be expressed by the host cell, e.g., as a fusion protein. In this context, the host cell is contacted with the protease substrate in the form of an expression vector capable of expressing the protease substrate. When it is desirable to determine the protease substrate, e.g., using a FRET approach, host cell culture lysate can be mixed with a peptide substrate and protease activity subsequently determined. Protease activity can be monitored intermittently, continuously or at a predetermined assay end point. According to the instant assay, viral protease activity correlates with infection kinetics. Thus, in embodiments using a FRET approach, viral protease activity and hence fluorescence is elevated in a cell infected with a virus, wherein a test agent that results in a disruption in the viral lifecycle will decrease protease activity (and decrease FRET fluorescence) as compared to an infected host cell culture not contacted with the test agent.

According to some embodiments, the host cell culture is contacted with the test agent before the host cell culture has been infected. In other embodiments, the host cell culture is contacted with the test agent during the exponential phase of viral spread through the host cell culture. The exponential phase of viral spread can be achieved using conditions and times known to provide exponential spread. Alternatively, the exponential phase of viral spread can be determined by experimentally monitoring the level of viral spread through the host cell culture, e.g., as determined by viral RNA levels. In still other embodiments, the host cell culture is contacted with the test agent at the time of infection or contact of the host cell culture with the virus.

Test agents which can be assayed in accordance with the present invention are generally derived from libraries of agents or compounds. Such libraries can contain either collections of pure agents or collections of agent mixtures. Examples of pure agents include, but are not limited to, proteins, polypeptides, peptides, antibodies, nucleic acids, oligonucleotides, carbohydrates, lipids, synthetic or semi-synthetic chemicals, and purified natural products. Examples of agent mixtures include, but are not limited to, extracts of prokaryotic or eukaryotic cells and tissues, as well as fermentation broths and cell or tissue culture supernates.

The assay of the invention can be performed in any format that allows rapid preparation and processing of multiple reactions. For example, stock solutions of the test agents as well as assay components can be prepared manually and all subsequent pipeting, diluting, mixing, washing, incubating, sample readout and data collecting carried out in a high throughput manner using commercially available robotic pipeting equipment, automated work stations, and analytical instruments for detecting the signal generated by the assay.

Because a central feature of the instant assay relates to the identification of inhibitors that target all steps in the viral lifecycle, the performance of the assay can be compared to standard RT-qPCR and western blot analyses to determine the equivalency between the assay methods. In addition, the assay can be validated by testing compounds that target entry, replication or egress. Since the instant assay is also highly compatible for measuring compound-mediated toxicity, particular embodiments further embrace assessing the cytotoxicity of test agent thereby facilitating the rapid identification and development of new and novel antiviral agents.

It is contemplated that agents identified by the assay of the invention can be used alone or in combination with other agents in methods for preventing and/or treating a viral infection. For therapeutic applications, desirably the agent is formulated for administration to a subject. In this respect, the agent can be combined in appropriate amounts in admixture with one or more pharmaceutically acceptable carriers. Such carriers are well-known in the art and include, e.g., saline solution, cellulose, lactose, sucrose, mannitol, sorbitol, and calcium phosphates. Optional additives include lubricants and flow conditioners, e.g., silicic acid, silicon dioxide, talc, stearic acid, magnesium/calcium stearates and polyethylene glycol (PEG) diluents; disintegrating agents, e.g., starch, carboxymethyl starch, cross-linked PVP, agar, alginic acid and alginates, coloring agents, flavoring agents and melting agents. Dyes or pigments may be added to tablets or dragee coatings, for example, for identification purposes or to indicate different doses of active ingredient.

Generally, the active ingredients are present in an amount of 1-95% by weight of the total weight of the composition. The composition may be provided in a dosage form that is suitable for the oral, parenteral (e.g., intravenously or intramuscularly), rectal, determatological, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Subjects benefiting from prevention and/or treatment with an agent of the invention include any animal (e.g., a mammal such as a human) susceptible to a viral infection disclosed herein. “Prevention” or “preventing” in the context of the present invention refers to prophylactic treatment which prevents or delays viral-associated clinical symptoms. In this respect, subjects benefiting from prophylactic treatment include, e.g., subjects suspected of being exposed to a virus, wherein prophylactic treatment prevents infection. In the context of the present invention, “treat” or “treating” refers to the administration of an antiviral agent to measurably slow or stop viral replication or spread, to measurably decrease the load of a virus, and/or to reduce at least one symptom associated with the viral infection. Desirably, the slowing of replication or decrease in viral load is by at least 20%, 30%, 50%, 70%, 80%, 90%, 95%, or 99%, as determined using a suitable assay (e.g., a replication assay or infection assay).

The dosage of an agent of the invention or a combination of agents depends on several factors, including the administration method, the type of virus to be treated, the severity of the infection, whether dosage is designed to treat or prevent a viral infection, and the age, weight, and health of the patient to be treated. An effective amount for use can be determined by a variety of means well known to those of skill in the art. For example, it is contemplated that one of skill in the art can choose an effective amount using an appropriate animal model system to test for inhibition of viral infection in vivo. The medical literature provides detailed disclosure on the advantages and uses of a wide variety of such models. Once a test drug has shown to be effective in vivo in animals, clinical studies can be designed based on the doses shown to be safe and effective in animals. One of skill in the art can design such clinical studies using standard protocols as described in textbooks such as Spilker ((2000) Guide to Clinical Trials. Lippincott Williams & Wilkins: Philadelphia).

The compounds disclosed herein are also useful tools in elucidating mechanistic information about the biological pathways involved in viral diseases. Such information can lead to the development of new combinations or single agents for treating, preventing, or reducing a viral disease. Methods known in the art to determine biological pathways can be used to determine the pathway, or network of pathways affected by contacting cells (e.g., hepatic cells) infected with a virus with the agents of the invention. Such methods can include, analyzing cellular constituents that are expressed or repressed after contact with the compounds of the invention as compared to untreated, positive or negative control compounds, and/or new single agents and combinations, or analyzing some other activity of the cell or virus such as an enzymatic activity, nutrient uptake, and proliferation. Cellular components analyzed can include gene transcripts, and protein expression. Suitable methods can include standard biochemistry techniques, radiolabeling the compounds of the invention, and observing the compounds binding to proteins, e.g., using 2D gels and/or gene expression profiling. Once identified, such compounds can be used in in vivo models (e.g., knockout or transgenic mice) to further validate the tool or develop new agents or strategies to treat viral disease.

The invention is described in greater detail by the following non-limiting examples.

### Example 1

### Materials and Methods for HCV Assay

Cells and Viruses. Huh7 cells (Zhong, et al. (2005) supra) were cultured in complete Dulbecco's Modified Eagle's Medium (cDMEM) (Hyclone, Logan, Utah) supplemented with 10% fetal bovine serum (FBS) (Hyclone), 100 units/ml penicillin, 100 mg/ml streptomycin, and 2 mM L-glutamine (Gibco Invitrogen, Carlsbad, Calif.) as previously described (Zhong, et al. (2005) supra).

The full-length JFH-1 genome is known under GENBANK Accession No. AB047639. The plasmid containing the full-length JFH-1 genome (pJFH1) has also been previously described (Kato, et al. (2003) Gastroenterology 125:1808-17; Kato, et al. (2001) supra; Wakita, et al. (2005) supra). Protocols for JFH-1 in vitro transcription and HCV RNA electroporation are routinely practiced in the art (Uprichard, et al. (2006) Virol. J. 3:89). The JFH-1 HCVcc viral stock was generated by infection of naïve Huh7 cells at a multiplicity of infection (MOI) of 0.01 focus forming units (FFU)/cell, using medium from Huh7 cells electroporated with in vitro transcribed JFH-1 RNA (Zhong, et al. (2005) supra).

Reagents. Recombinant human interferon-α 2a (IFN-α 2a), IFN-β and IFN-γ (PBL Biomedical Laboratories, New Brunswick, N.J.) were resuspended to a concentration of 50 U/μl in complete DMEM supplemented with 10% FBS, aliquoted into single use tubes, and stored at −80° C. Cyclosporin A (CsA; Nakagawa, et al. (2004) Biochem. Biophys. Res. Commun. 313:42-7) and Naringenin (Nahmias, et al. (2008) Hepatology 47:1437-45) were purchased from Sigma (St. Louis, Mo.) and resuspended to concentrations of 10 mM and 50 mM, respectively, in DMSO (Sigma). Mycophenolic acid (MA; Henry, et al. (2006) Gastroenterology 131:1452-62) (Sigma) was resuspended to a concentration of 50 mM in 95% ETOH. N-butyldeoxynojirimycin (NB-DNJ; Steinmann, et al. (2007) Hepatology 46:330-8) (Sigma) was resuspended to a concentration of 25 mM in dH₂O. The nucleoside polymerase inhibitor NM107 (Mathy, et al. (2008) Antimicrob. Agents Chemother. 52:3267-75) was resuspended to a concentration of 10 mM in DMSO (Sigma). Reagents were aliquoted into single use tubes, and stored at −20° C. Anti-HCV E2 monoclonal antibody (C1) has been previously described (Law, et al. (2008) Nat. Med. 14:25-7; Zhong, et al. (2005) supra). The anti-human CD81 monoclonal antibody was purchased from Serotec (Raleigh, N.C.). Recombinant HCV NS3/4A protease was purchased from AnaSpec (San Jose, Calif.). When added to cells, all reagents were diluted to a specific concentration in cDMEM containing a final DMSO concentration of 1%. Although inhibitor concentrations chosen were in part determined based on previously published reports, it is relevant to note that these past studies were generally conducted using HCV subgenomic replicons of varying genotypes in actively dividing cells and thus the reported IC₅₀s cannot be directly compared.

The 5-FAM/QXL™520 NS3 FRET substrate (Anaspec), modeled upon the NS4A/NS4B site-derived (Asp-Glu-Met-Glu-Glu-Cys-Ala-Ser-His-Leu; SEQ ID NO:2) depsipeptide substrate (Bianchi, et al. (1996) supra), is an internally quenched peptide with a fluorescent donor (5-Carboxyfluorescein, 5-FAM) and acceptor (QXL) on opposing sides of the NS3 protease cleavage site. The donor absorbs energy at 490 nm and emits energy (i.e., fluorescence) at 520 nm. However, when in close contact on an intact peptide, the acceptor absorbs the 520 nM energy emitted by the donor, thereby preventing fluorescence. Cleavage of the peptide increases the distance between the fluorophores resulting in proportional 5-FAM fluorescence. This NS3 FRET substrate allows for enzymatic assays to be performed at high wavelengths providing increased fluorescence quantum yield, diminished auto fluorescence (commonly detected with other fluorophores, such as EDANS) and more sensitivity than other NS3 FRET substrates (Fattori, et al. (2000) J. Biol. Chem. 275:15106-13; Kakiuchi, et al. (1999) supra; Konstantinidis, et al. (2007) Anal. Biochem. 368:156-67; Mao, et al. (2008) Anal. Biochem. 373:1-8; O′Boyle, et al. (2005) supra) allowing for the detection of as little as 0.1 pmole of HCV NS3 protease.

Non-HTS HCV Infection Kinetics Assay. Huh7 cells were seeded at 7×10⁴ cells in each well of a 12-well plate (BD Biosciences, San Jose, Calif.). Twenty-four hours post-seeding, cells were infected with JFH-1 HCVcc at a MOI of 0.01 FFU/cell in a total volume of 1 ml cDMEM. Throughout the course of the experiment, infected cells were trypsinized just before reaching confluence and re-plated at a dilution of 1:3 to maintain active cell growth. At indicated times post-infection, medium was harvested from wells for infectivity titration analysis, RNA was isolated from triplicate wells for reverse transcription followed by real-time quantitative PCR(RTqPCR) analysis, and protein was isolated for western blot analysis.

RNA Isolation and RT-qPCR Analysis. Total cellular RNA was isolated using a 1× Nucleic Acid Purification Lysis Solution (Applied Biosystems, Foster City, Calif.) and purified using an ABI PRISM™ 6100 Nucleic Acid PrepStation (Applied Biosystems), as per the manufacturer's instructions. One μg of purified RNA was used for cDNA synthesis using the TAQMAN reverse transcription reagents (Applied Biosystems), followed by SYBR green RT-qPCR using an Applied Biosystems 7300 real-time thermocycler (Applied Biosystems). Thermal cycling included of an initial 10-minute denaturation step at 95° C. followed by 40 cycles of denaturation (15 seconds at 95° C.) and annealing/extension (1 minute at 60° C.). HCV JFH-1 and GAPDH transcript levels were determined relative to a standard curve derived from serial dilutions of plasmid containing the JFH-1 HCV cDNA or the human GAPDH gene, respectively. The PCR primers used to detect GAPDH and HCV were: human GAPDH (GENBANK Accession No. NM 002046) sense, 5′-GAA GGT GAA GGT CGG AGT C-3′ (SEQ ID NO:3) and anti-sense, 5′-GAA GAT GGT GAT GGG ATT TC-3′ (SEQ ID NO:4); and JFH-1 HCV (GENBANK Accession No. AB047639) sense, 5′-TCT GCG GAA CCG GTG AGT A-3′ (SEQ ID NO:5) and anti-sense, 5′-TCA GGC AGT ACC ACA AGG C-3′ (SEQ ID NO:6).

Extracellular Infectivity Titration Assay. Cell supernatants were serially diluted 10-fold in cDMEM and 100 μl was used to infect, in triplicate, 4×10³ naïve Huh7 cells per well in 96-well plates (BD Biosciences). The inoculum was incubated with cells for 24 hours at 37° C. and then overlaid with 150 μl complete DMEM containing 0.4% methylcellulose (w/v) (Fluka BioChemika, Switzerland) to give a final concentration of 0.25% methylcellulose. Seventy-two hours post-infection, medium was removed, cells were fixed with 4% paraformaldehyde (Sigma) and immunohistochemical staining for HCV E2 was performed. Briefly, cells were incubated with 1×PBS containing 0.3% (v/v) hydrogen peroxide (Fisher, Fairlawn, N.J.) to block endogenous peroxidase. Following three rinses with 1×PBS, cells were blocked for 1 hour with 1×PBS containing 0.5% (v/v) TRITON X-100 (Fisher), 3% (w/v) bovine serum albumin (BSA) (Sigma) and 10% (v/v) FBS. The HCV E2 glycoprotein was detected by incubation at room temperature with 1×PBS containing 0.5% (v/v) TRITON X-100 and 3% (w/v) BSA and a 1:500 dilution of the human monoclonal anti-HCV E2 antibody C1. Bound C1 was subsequently detected by a 1 hour incubation with a 1:1000 dilution of an HRP-conjugated anti-human antibody (Pierce, Rockford, Ill.) followed by a 30 minute incubation with an AEC (3-amino-9-ethylcarbazole) detection substrate (BD Biosciences). Cells were washed with dH₂O and visualized using a ZEISS AXIOVERT microscope (Carl Zeiss, Germany). Viral infectivity titers are expressed as FFU per milliliter of supernatant (FFU/ml), determined by the average number of E2-positive foci detected in triplicate samples at the highest HCV-positive dilution.

Western Blot Analysis. Cells were harvested in 1.25% TRITON X-100 lysis buffer (TRITON X-100, 50 mM Tris-HCl, pH 7.5, 150 mM NaCl, 2 mM EDTA) supplemented with a protease inhibitor cocktail (Roche Applied Science, Indianapolis, Ind.). Fifty micrograms of protein was resolved by SDS-PAGE and transferred to HYBOND nitrocellulose membranes (Amersham Pharmacia, Piscataway, N.J.). Membranes were sequentially blocked with 5% non-fat milk, incubated with a 1:1000 dilution of either a monoclonal mouse anti-HCV NS3 antibody (Clone 9-G2; ViroGen, Watertown, Mass.) or a monoclonal mouse anti-HCV Core antibody (Clone C₇₋₅₀; Affinity BioReagents, Rockford, Ill.), washed 3 times with PBS/0.05% TWEEN 20, incubated with horseradish peroxidase-conjugated goat anti-mouse antibody (Pierce, Rockford, Ill.), and washed again. Bound antibody complexes were detected with SUPERSIGNAL chemiluminescent substrate (Pierce).

High-Throughput HCVcc FRET Assay. Huh7 cells were seeded in 96-well BIOCOAT culture black plates with clear bottoms (BD Biosciences) at a density of 8×10³ cells/well in cDMEM. Upon reaching 90% confluence, media was replaced with 200 μl cDMEM supplemented with 1% DMSO (Sigma), and cells were cultured for an additional 20 days, replacing medium every 2 days as previously described (Choi, et al. (2009) supra; Sainz, Jr. & Chisari (2006) supra). For inhibition experiments, cultures were inoculated with HCVcc JFH-1 at an MOI of 0.05 FFU/cell. Unless otherwise indicated, uninfected and infected cultures were mock treated or treated with specified compounds at 0, 2 and 4 days post-infection. On day six post-infection, medium was removed from culture plates and cells were lysed in 50 μl 1.25% TRITON X-100 lysis buffer (TRITON X-100, 50 mM Tris-HCl, pH 7.5, 150 mM NaCl, 2 mM EDTA) and immediately frozen (−80° C.). Initially, a panel of lysis buffers and conditions were rigorously tested to determine the optimal parameters to ensure maximum lysis with minimum non-specific FRET background. Based on these analyses, it was concluded that cultures could be immediately lysed using 1.25% TRITON X-100 lysis buffer, without washing, after removal of phenol-red containing cDMEM.

For FRET analysis, plates were brought to room temperature and then placed in a FLUOstar OPTIMA microplate reader (BMG Labtech, Durham, N.C.), which automatically injects into each well 50 μl of the 5-FAM/QXL™520 NS3 FRET substrate (Anaspec), diluted to a final concentration of 5 μM. 5-FAM dequenching was measured at 490 nm (excitation) and 520 nm (emission) for 20 cycles in kinetic mode. Reported relative fluorescence units (RFU) were determined by endpoint analysis of RFU at approximately cycle 20, the cycle determined to give the maximum signal-to-noise ratio (i.e., 6).

Cytotoxicity Assay. As an important secondary screen for cytotoxicity, the TOXILIGHT BioAssay Kit (Lonza, Walkersville, Md.), a bioluminescence-based assay which measures adenylate kinase (AK) released from damaged cells, was used to assess drug-induced cellular toxicity, as per the manufacturer's instructions. Briefly, 20 μl of supernatant was collected on day six post-infection and transferred to white 96-well plates (BD Biosciences). One hundred μl of adenylate kinase detection reagent was then added to each well, and luminescence, expressed as relative light units (RLU), was measured (FLUOstar OPTIMA).

Calculations. All RFU and RLU values were background subtracted (1.25% TRITON X-100 lysis buffer alone or medium alone, respectively). RFU values from non-treated HCV-infected wells and RLU values from mock-treated wells were considered as 100% maximum activity. Signals from other wells are expressed as a percentage of the appropriate maximum. The Z′ was calculated using the equation 1-[(3σ_{c+}+3σ_{c-})/(μ_{c+}-μ₋)], where 3σ_{c+} is the standard deviation of the signal (non-treated), 3σ_{c+} is the standard deviation of the background (treated), μ_{c+} is the average RFU of the signal (non-treated) and μ_{c-} is the average RFU of the background (treated).

### Example 2

### HCV NS3 Protein Levels Parallel HCVcc Infection Kinetics

NS3 protease activity has been shown to be an accurate readout for HCV replication in replicon-based cell culture systems, providing the same EC₅₀ calculation for IFN-β inhibition as that obtained by RT-qPCR analysis of replicon RNA (O′Boyle, et al. (2005) supra). To determine if viral protein levels could also be used to monitor HCVcc infection, the kinetics of HCV protein accumulation in Huh7 cells infected with HCVcc at an MOI of 0.01 FFU/cell were assessed by western blot analysis and compared to HCV RNA expansion and de novo production of infectious HCVcc. The results of this analysis indicate that HCV protein levels, in particular NS3, correlate well with HCV RNA levels and infectious virus production indicating that it is possible to use NS3 protease activity as a virally encoded “enzymatic reporter” of HCVcc infection, rather than using a genetically engineered HCVcc encoding an exogenous reporter such as luciferase (Koutsoudakis, et al. (2006) J. Virol. 80:5308-20; Tscherne, et al. (2006) J. Virol. 80:1734-41; Zhang, et al. (2008) supra).

### Example 3

### HCV NS3 FRET Signal Increases Linearly with Intracellular NS3 Protein Levels

To verify the feasibility of using FRET to quantitatively measure HCV NS3 protein levels, it was initially confirmed that NS3-dependent cleavage of an internally quenched peptide substrate would produce a FRET signal linear with the amount of NS3 present. Purified recombinant NS3/4A protease was serially-diluted, incubated with the 5-FAM/QXL™520 NS3 FRET peptide substrate, and NS3 FRET activity was measured. The results of this analysis indicated that FRET signal increased with increasing amounts of purified NS3, revealing a linear correlation (R²=0.999) between NS3 protein levels and FRET activity. To determine if such a linear correlation could be achieved with intracellular NS3 protein, NS3 FRET activity was determined using lysates from serially diluted sgJFH-1 replicon cell or Huh7 cells infected with HCVcc JFH-1 at increasing MOIs (0.05, 0.10, 0.50 and 1.0 FFU/cell). Similar to the results obtained using purified NS3/4A protease, the FRET assay carried out with lysates from sgJFH-1 replicon cells or Huh7 cells infected with HCVcc JFH-1 exhibited a linear signal (R²=0.996 and R²=0.999, respectively) relative to intracellular NS3 protease concentrations.

### Example 4

### Cell-Based HCVcc Infection Assay

Having identified a suitable assay readout, the optimal cell culture conditions necessary for a cell-based HCVcc infection HTS assay were determined. Since cell culture variability and non-specific effects of compounds on cell growth can be a problem for cell-based HTS, particularly for HCV-based assays where confluence and changes in the state of the host cell can have a negative affect on viral replication (Nelson & Tang (2006) J. Virol. 80:1181-90; Pietschmann, et al. (2001) J. Virol. 75:1252-1264; Sainz, Jr. & Chisari (2006) supra; Windisch, et al. (2005) J. Virol. 79:13778-93), non-dividing Huh7 cells were selected for the cell-based HCVcc infection assay. As previously described (Sainz, Jr. & Chisari (2006) supra), treatment of Huh7 cells with 1% DMSO for 20 days induces cell growth arrest allowing non-dividing, HCV-permissive Huh7 cells to be maintained at a stable cell number for extended periods of time (>100 days). In this respect, when replicate 96-well cultures of non-dividing, G₀ synchronized Huh7 cells were infected with HCVcc at a MOI of 0.05 FFU/cell, high reproducibility between wells was observed in HCV NS3 protein (FRET) and RNA (RT-qPCR) at day six post-infection, and de novo infectious virus titers achieved at days 3, 5, 7, and 25 post-infection. Therefore, this cell system minimizes the well-to-well variability commonly associated with cell-based HTS assays which typically use rapidly dividing, unsynchronized cell cultures.

To determine assay conditions under which HCV NS3 protease activity can be used to quantitatively assess HCVcc infection progression, the kinetics of NS3 protease activity in DMSO-treated Huh7 cells was assessed after infection with increasing MOIs of HCVcc. Specifically, non-dividing cultures of Huh7 cells were infected with HCVcc at an MOI of 0.01, 0.05, or 0.1 FFU/cell and HCV RNA levels and NS3 protease activity were measured daily for 10 days by RT-qPCR and FRET, respectively. This analysis indicated that HCV RNA levels increased exponentially from day one to day eight post-infection in a MOI-dependent manner, reaching steady state levels of ˜1×10⁷ copies/μg RNA by day six-to-eight post-infection. Likewise, HCV NS3 protease activity, as determined by 5-FAM dequenching, also demonstrated a steady increase through day 10 post-infection, and then, like HCV RNA levels, remained at a constant plateau level at later time points examined. Similar to HCV RNA expansion, a linear increase in HCV NS3 protease activity up to day eight post-infection at MOIs of 0.01 and 0.05 (R²=0.999 and 0.989, respectively) was observed. When plotted as a function of one another, a linear correlation between HCV RNA expansion and NS3 protease activity was observed (R² value=0.983), confirming that HVC NS3 protease activity directly parallels HCV RNA expansion over an extended period within which quantitative end-point HTS analyses of HCVcc infection can be performed.

### Example 5

### Low MOI HCVcc HTS FRET Assay can Quantitatively Identify Inhibitors that Target any Aspect of the HCV Lifecycle

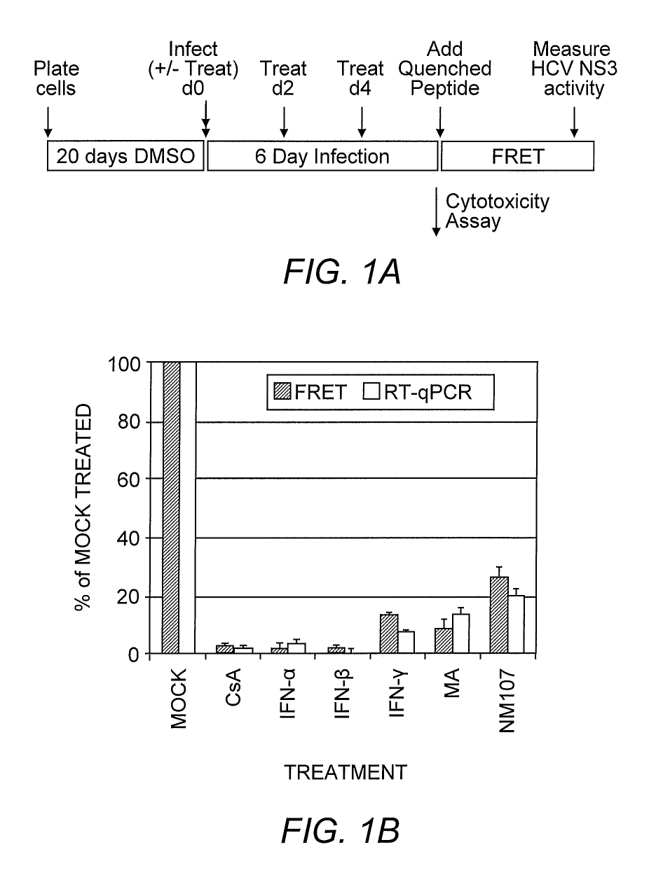

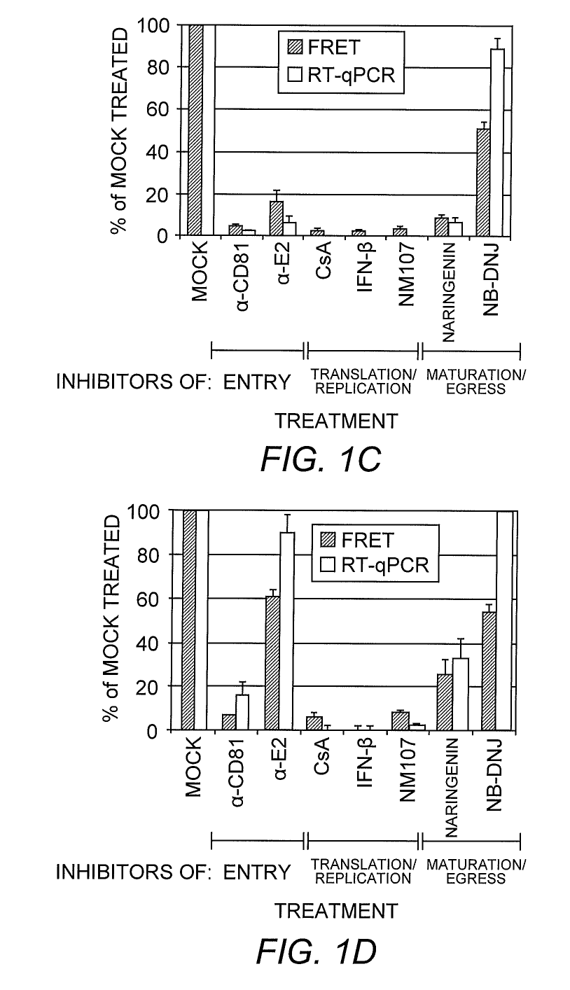

Based on the ability to reproducibly perform low MOI HCV infection over several days in non-dividing Huh7 cells, a novel HCV infection HTS assay was designed whereby compounds were added during the exponential phase of HCV spread throughout the culture with NS3 protease activity being assessed at day six post-infection after multiple rounds of infection and re-infection. The rationale being that, unlike studies which are limited to a single cycle of virus replication, inhibitors that target any aspect of the viral lifecycle (e.g., entry, replication, assembly, egress and spread) should be detectable using a low MOI approach. To validate this HTS experimental design (FIG. 1A), the ability of the cell-based FRET assay to identify inhibitors of HCVcc was compared to that of standard RT-qPCR and western blot analyses. In addition, it was also confirmed that the low MOI, six-day experimental strategy could effectively detect inhibitors that target any aspect of the viral lifecycle.

Using known HCV inhibitors (Henry, et al. (2006) supra; Mathy, et al. (2008) supra; Nakagawa, et al. (2004) supra; Zhong, et al. (2005) supra), it was determined whether the HCV NS3 FRET assay was able to identify HCV inhibitors analogous to non-HTS assays such as RT-qPCR (FIG. 1B) and western blot. For this analysis, IFN-α, -β and -γ, and three HCV replication inhibitors, the immunosuppressive drugs CsA (Nakagawa, et al. (2004) supra) and MA (Henry, et al. (2006) supra), and the HCV-specific nucleoside polymerase inhibitor NM107 (Mathy, et al. (2008) supra) were tested. In the case of IFN-α, -β and CsA, the NS3 FRET assay and RT-qPCR indicated over 98% inhibition. Likewise, both assays measured a comparable 74-93% inhibition range for IFN-γ, MA and NM107. While less quantitative in nature, western blot analysis of NS3 proteins levels also paralleled the NS3 FRET protease activity detected, demonstrating equivalency between the HCV NS3 FRET assay and standard analyses to accurately identify HCV inhibitors at the level of percent inhibition.

To confirm that the low MOI (e.g., 0.05 FFU/cell), six-day infection strategy would effectively identify inhibitors that target any aspect of the viral lifecycle (e.g., entry, replication, assembly, egress and spread), HCV NS3 activity was measured following treatment of cells with a panel of HCV antivirals shown to target different aspects of the viral lifecycle (Henry, et al. (2006) supra; Mathy, et al. (2008) supra; Nahmias, et al. (2008) supra; Nakagawa, et al. (2004) supra; Steinmann, et al. (2007) supra; Zhong, et al. (2005) supra). As illustrated in FIG. 1A, DMSO-Huh7 cells were infected with HCV at 0.05 FFU/cell and compounds were added either at the time of infection (co-) or two days post-infection, replenished every two days over the six day assay, and HCV RNA and NS3 protein levels were measured by RT-qPCR and by FRET, respectively, six days post-infection. When added either at the time of infection (FIG. 1C) or 2 days post-infection (FIG. 1D) all inhibitors tested efficiently reduced both HCV RNA replication and NS3 protease activity to equivalent levels. However, the affect of inhibitors that targeted HCV entry (i.e., α-CD81 and α-E2) was less pronounced when added post-infection (FIG. 1D).

In addition, since secondary toxicity screens are a necessary component of any HTS campaign, a luminescence-based cellular toxicity assay (TOXILIGHT®, Lonza) was incorporated into the assay to assess compound-mediated cytotoxicity. Since this assay quantitatively measures adenlyate kinase release into the culture medium from damaged cells, cellular toxicity and FRET can be measured from the same well by simply removing 20 μl of the culture medium prior to cell lysis (FIG. 1A). This assay confirmed that none of the compounds tested exhibited any non-specific cytotoxic affect, as compared to a positive control culture treated with 10% TRITON X-100. Thus, these data together demonstrate the utility of this cell-based HCVcc HTS assay for identifying inhibitors that target all aspects of the viral lifecycle and the compatibility of the assay design for assessing compound-mediated cytotoxicity.

### Example 6

### Statistical Validation of HCV FRET Assay Performance

The quality of a HTS assay can be determined according to its primary goal, which is to distinguish hits from non-hits. The Z′ statistic is a measure of the distance between the standard deviations for the positive (signal) and negative (noise) controls of the assay. This value reflects not only the size of the window between the positive and negative controls, but also assesses the noise/error associated with the control assays. To determine the Z′ value of the cell-based FRET assay herein, full plates containing un-treated and treated samples were analyzed. IFN-β was used as a positive control inhibitor of HCV replication and the Z′ was calculated as described herein. The data for three representative plates were graphically plotted and the respective Z′ values of 0.604, 0.643 and 0.654 were obtained for each plate with an average signal-to-background ratio of seven. Similar Z′ values (i.e., >0.5) were obtained when CsA was used as alternate inhibitor. Taken together, these data indicate an acceptable signal-to-noise window and therefore satisfy the criteria for an HTS assay.

### Example 7

### HIV Protease, Substrates and Infection of Non-Dividing Host Cells

HIV-1 protease (HIV PR) is an aspartic protease that is essential for the life-cycle of HIV. HIV PR is required for the post-translational cleavage of the precursor polyproteins, Pr^{gag} and Pr^{gag-pol} (Seelmeier, et al. (1988) Proc. Natl. Acad. Sci. USA 85:6612-6616). These cleavages are essential for the maturation of HIV infectious particles; without effective HIV PR, HIV virions remain uninfectious (Kräusslich, et al. (1989) Proc. Natl. Acad. Sci. USA 86(3):807-11; Kohl, et al. (1988) Proc. Natl. Acad. Sci. USA 85(13):4686-90).

Peptide substrates of HIV PR are known in the art and include, but are not limited to, Lys-Ala-Arg-Val-Leu-Ala-Glu-Ala-Met (SEQ ID NO:7), Arg-Gln-Ala-Asn-Phe-Leu-Gly-Lys (SEQ ID NO:8), Ser-Gln-Asn-Tyr-Pro-Ile-Val-Gln (SEQ ID NO:9), or derivatives thereof. See You, et al. (2005) J. Virol. 79:12477-12486 and references cited therein.

Human immunodeficiency virus type 1 (HIV-1) replicates efficiently in both non-dividing (post-mitotic) and dividing cells (Weinberg, et al. (1991) J. Exp. Med. 174:1477-1482; Lewis, et al. (1992) EMBO J. 11:3053-3058). HIV-1 infection of non-dividing cell populations in vivo, and in particular macrophages and mucosal dendritic cells, appears to be essential for initiating a pathogenic infection and for establishing viral reservoirs that can persist for extended periods (Gartner, et al. (1986) Science 233:215-219; Koenig, et al. (1986) Science 233:1089-1093; Wiley, et al. (1986) Proc. Natl. Acad. Sci. USA 83:7089-7093; Schuitmaker, et al. (1992) J. Virol. 66:356-363).

Thus, in accordance with embodiments drawn to the identification and anti-HIV agents, non-dividing monocyte-derived macrophages (MDMs) and primary blood lymphocytes (PBLs) can be employed. MDMs and PBLs can be derived from the peripheral blood of healthy volunteer donors following venepuncture. MDMs can be purified by gelatin-coated plastic adherence (Collman, et al. (1989) J. Exp. Med. 170:1149-1163) and maintained in culture dishes at a density of 4×10⁵ cells per well in DMEM supplemented with fetal bovine serum and recombinant human granulocyte macrophage colony-stimulating factor (rhGM-CSF) for 7 days prior to viral challenge. PBLs are purified using FICOLL-Paque, stimulated with phytohemagglutinin (PHA) for 72 hours and maintained in RPMI 1640 medium containing fetal bovine serum and recombinant interleukin-2 (rIl-2) at a density of ˜1×10⁶ cells/ml (Simon & Malim (1996) J. Virol. 70:5297-5305).

Stocks of wild-type HIV-1 (e.g., isolate YU-2) are generated by transient calcium phosphate-mediated transfection of 293T cultures. At 24 hours, the supernatants are harvested and stored in aliquots at −80° C. Upon challenge of MDMs or PBLs with wild-type HIV-1, cultures are maintained by replenishing the culture media at 2- to 3-day intervals.

### Example 8

### Dengue Virus Protease, Substrates and Infection of Non-Dividing Host Cells

Analysis of polyprotein processing establishes that the NS3 protease of Flaviviruses (e.g., Dengue virus, West Nile virus and Yellow fever) plays a key role in the lifecycle of these viruses. For example, the Dengue virus NS3 protease catalyzes the cleavage of NS2A-NS2B, NS2B-NS3, NS3-NS4A, and NS4B-NS5 sites in the polyprotein which have Lys-Arg, Arg-Arg, Arg-Lys, and occasionally Gln-Arg at the P2 and P1 positions, followed by a short chain amino acid Gly, Ala, or Ser at the P1′ position (Chambers, et al. (1993) J. Virol. 67:6797-6807; Arias, et al. (1993) supra; Chambers, et al. (1990) Proc. Natl. Acad. Sci. USA 87:8898-8902; Zhang, et al. (1992) J. Virol. 66:7549-7554; Preugschat, et al. (1990) J. Virol. 64:4364-4374; Falgout, et al. (1991) J. Virol. 65:2467-2475; Chambers, et al. (1991) J. Virol. 65:6042-6050).

Peptide substrates of the Dengue virus NS3 protease are known in the art and include, but are not limited to, Arg-Thr-Asn-Lys-Lys-Arg-Ser-Trp-Pro-Leu-Asn-Glu (SEQ ID NO:10), Glu-Val-Lys-Lys-Gln-Arg-Ala-Gly-Val-Leu-Trp-Asp (SEQ ID NO:11), Phe-Ala-Ala-Gly-Arg-Lys-Ser-Leu-Thr-Leu-Asn-Leu (SEQ ID NO:12), Thr-Thr-Asn-Thr-Arg-Arg-Gly-Thr-Gly-Asn-Ile-Gly (SEQ ID NO:13), Lys-Gly-Ala-Ser-Arg-Arg-Ser-Trp-Pro-Leu-Asn-Glu (SEQ ID NO:14), and Gln-Val-Lys-Thr-Gln-Arg-Ser-Gly-Ala-Leu-Trp-Asp (SEQ ID NO:15).

The liver is indicated as a major target of dengue virus infection. In this respect, Huh-7 cells have been shown to be susceptible to dengue virus infection (Lin, et al. (2000) J. Med. Virol. 60(4):425-31). Accordingly, inhibitors of dengue virus infection can be identified using Huh7 cells, as described herein for HCV.

Stocks of dengue virus, e.g., the Hawaii (DEN-1), New Guinea (DEN-2), H-87 (DEN-3), or H-241 (DEN-4) strain, can be obtained from culture supernatants of infection of mosquito C6/36 cells and then titrated on BHK-21 cells by standard plaque-forming assay.

### Example 9

### Rhinovirus Protease, Substrates and Infection of Non-Dividing Host Cells

Human rhinoviruses (HRVs) are picornaviruses that constitute the major causative agent of the common cold in humans (Gwaltney, Jr. (1982) In: Viral Infection of Man: Epidemiology and Control. Evans (Ed.) 2^{nd} Ed., Plenum Publishing Corp., New York, N.Y., pp. 491-517). Other members of the picornavirus family are also human pathogens and include the enteroviruses: poliovirus type I, hepatitis A, and coxsackie B viruses. As with all picornaviruses, the positive strand RNA genome of rhinoviruses is translated directly into a large viral polyprotein precursor which undergoes a series of controlled proteolytic cleavages to generate functional viral gene products.

Studies with the 3C protease of HRV14 have indicated that the substrate requirements of the enzyme are satisfied by authentic HRV cleavage sites which contain a Gln/Gly scissile bond (Cordingley, et al. (1989) J. Virol. 63:5037-5045; Orr, et al. (1989) J. Gen. Virol. 70:2931-2942). Sequence analysis further indicated that Thr-Leu-Phe-Gln-Gly-Pro (SEQ ID NO:16) is the minimal substrate recognized and cleaved by the HRV14 3C protease (Cordingley, et al. (1990) J. Biol. Chem. 265:9062-9065). Asp-Val-Met-Thr-Ala-Ile-Phe-Gln-Gly-Pro-Ile-Asp-Met-Lys-Asn-Pro (SEQ ID NO:17), containing the Gln/Gly scissile bond, is also a suitable peptide substrate for serotype HRV2 and HRV14 3C proteases (Cordingley, et al. (1990) supra).

Normal human bronchial epithelial cells (NHBE; Clonetics Corp., Walkersville, Md.) are known to be susceptible to infection with HRV (Whiteman, et al. (2003) J. Biol. Chem. 278:11954-11961). NHBE cells can be cultured in small airway basal medium (SABM) or bronchial epithelial cell growth medium (BEGM) at 37° C. in humidified air containing 5% CO₂ according to standard protocols (Whiteman, et al. (2003) supra). To induce blockage of G2/M cell cycle progression, NHBE cells can be grown in the presence of zinc. For example, supplementation of zinc-free BEGM with 32 μM ZnSO₄ can be used to produce non-dividing NHBE cells (Shih, et al. (2008) Exp. Biol. Med. 233(3):317-27) for use in accordance with the instant method.

HRV (e.g., HRV16, HRV14, HRV2, or HRV1A) can be grown and titered in HeLa cells according to conventional methods (Mosser, et al. (2002) J. Infect. Dis. 185:734-743; Sethi, et al. (1997) Clin. Exp. Immunol. 110:362-369). Briefly, confluent monolayers of HeLa cells are inoculated with a known dilution (e.g., 10^{2.5}, TCID₅₀/ml) of HRV and incubated for 90 minutes at 34° C. in humidified air containing 5% CO₂, after which, cells are cultured until the cytopathic effect (CPE) is >80%. Medium containing virus is centrifuged at 600×g for 10 minutes, after which the viral suspension is stored at −80° C.

For infection, bronchial epithelial cell suspensions are centrifuged and resuspended in PBS containing calcium, magnesium and HRV at a low MOI. After a 30-90 minute incubation at room temperature for viral attachment, medium is added to cell suspensions containing HRV and cells are incubated for an additional period of time, e.g., 8 hours (34° C., 5% CO₂) for viral replication.

### Example 10

### Norwalk virus Protease, Substrates and Infection of Non-Dividing Host Cells

Norwalk virus is a member of the Norovirus genus of the viral family Caliciviridae. Noroviruses are the major causative agents of nonbacterial, acute gastroenteritis in humans. The Norwalk virus (NV) genome is a positive sense, single-stranded RNA that encodes three open reading frames. Similar to the case in picornaviruses, the Norwalk virus protease (NV^{PRO}) is necessary to cleave its viral polyprotein into the six nonstructural proteins (Blakeney, et al. (2003) Virology 308:216-224) required for viral maturation and replication.

Primary cleavage sites in the ORF1 polyprotein of two Norwalk-like viruses have been identified as Gln/Gly dipeptides (Hardy, et al. (2002) Virus Res. 89:29-39). An exemplary peptide substrate for NV^{PRO} includes, but are not limited to, Glu-Pro-Asp-Phe-His-Leu-Gln-Gly-Pro-Glu-Asp-Leu-Ala-Lys (SEQ ID NO:18)(Zeitler, et al. (2006) J. Virol. 80:5050-5058), corresponding to the cleavage site between p48 and p41 in the polyprotein.

Transfection of NV RNA, isolated from stool samples, into human hepatoma Huh-7 cells has been shown to lead to viral replication, with expression of viral antigens, RNA replication, and release of viral particles into the medium (Guix, et al. (2007) J. Virol. 81:12238-12248). Accordingly, inhibitors of NV infection can be identified using Huh7 cells, as described herein for HCV.

NV can be isolated form stool samples using conventional methods (Guix, et al. (2007) supra). Briefly, stool suspensions in PBS are extracted with VERTREL XF and centrifuged at 12,400×g for 10 minutes. The supernatant is collected, and virus is precipitated by adding polyethylene glycol-NaCl solution and incubating the mixture for 2 hours at 4° C. The precipitated virus is pelleted and purified by isopycnic CsCl gradient centrifugation. After gradient fractionation, viruses are recovered by ultracentrifugation and presence of virus in each fraction is analyzed by, e.g., enzyme-linked immunosorbent assay (ELISA) specific for the NV VP1 capsid protein, quantitative real-time reverse transcription-PCR, and/or electron microscopy according to known methods. Isolation of RNA from the peak fraction containing NV is performed using, e.g., the QIAAMP viral RNA mini kit (QIAGEN). RNA is subsequently transfected into cells according to conventional protocols (Guix, et al. (2007) supra).

### Example 11

### Rubella Virus Protease, Substrates and Infection of Non-Dividing Host Cells

The genomic RNA of rubella virus, the causative agent of the measles, contains two long open reading frames (ORF), the 5′ proximal nonstructural-protein ORF (NSP-ORF), encoding nonstructural proteins involved in viral RNA replication, and the 3′ proximal ORF, encoding the virion proteins (Frey (1994) Adv. Virus Res. 44:69-160). Following translation of the NSP-ORF from the genomic RNA, a papain-like cysteine protease within the NSP-ORF sequences cleaves the precursor (P200) into two mature products, P150 (150 kDa) and P90 (90 kDa), which are N- and C-terminal within the ORF, respectively. The cleavage site of the Rubella protease has been shown to be between G₁₃₀₁ and G₁₃₀₂ of P200 (Chen, et al. (1996) J. Virol. 70:4707-4713; Marr, et al. (1994) Virology 198:586-592; Pugachev, et al. (1997) Arch Virol. 142:1165-1180). In this respect, an exemplary peptide substrate of the Rubella protease includes, but is not limited to, Ser-Arg-Gly-Gly-Gly-Thr-Cys-Ala (SEQ ID NO:19).

Rubella virus can infect non-dividing human normal-term placenta chorionic villi explants (CVE) and monolayers of cytotrophoblasts (CTB) (Adamo, et al. (2004) Viral Immunol. 17(1):87-100). CTB cells are of particular interest in that transformed, cell-contact, growth-inhibited CTB cells lines available in the art (i.e., the cells stop growing when confluence is reached). In addition, the human MCF-7 cell line (American Type Culture Collection, Manassas, Va.) is susceptible to infection with the DBS strain of rubella virus at a low multiplicity of infection (Williams, et al. (1981) J. Gen. Virol. 52:321-328; Roehrig, et al. (1979) J. Virol. 29:417-420). MCF-7 cells can be maintained in culture using conventional methods. Briefly, MCF-7 cells are grown in DMEM supplemented with fetal calf serum, insulin and amino acid concentrate. MCF-7 cells can be passaged at one-week intervals and arrested in G0 by treatment with anti-estrogens such as ICI 182,780 (Doisneau-Sixou, et al. (2003) Endocrine-Related Cancer 10:179-186).

Virus stocks of rubella are prepared by infection of RK-13 cells respectively. Virus titers are determined by plaque assay in the same cell lines. Plaque formation by rubella virus requires the use of an overlay composed of McCoy 5A medium containing 2% fetal bovine serum and 0.5% agarose. At the appropriate time, the agarose overlay is removed, the cells stained with neutral red and the plaques counted.

### Example 12

### SARS Coronavirus Protease, Substrates and Infection of Non-Dividing Host Cells

Examination of the SARS coronavirus sequences reveals that the rep gene covers over 20,000 nucleotides and encodes two overlapping polyproteins. Viral entry into the cell is followed by translation of the viral rep gene, which codes for a viral protease within the polyprotein, Mpro or 3CLpro. The SARS 3CLpro has also been verified in vitro to cleave after the Gln residue at Leu-Gln-(Ser, Ala, Gly). Polypeptides released from the polyproteins by the main viral protease Mpro or 3CLpro include the viral polymerase and a protease. Both products are essential for viral replication and transcription.

Investigations of substrate specificity of SARS CoV Mpro indicate that the octapeptides with sequences of Ser-Ala-Val-Leu-Gln-Ala-Gly-Phe (SEQ ID NO:20) and Thr-Val-Lys-Leu-Gln-Ser-Gly-Phe (SEQ ID NO:21) are optimal for cleavage (Fan, et al. (2005) Biochem. Biophys. Res. Commun. 329:934-40).

Differentiated adult human alveolar type II cells and Vero E6 Cells (American Type culture Collection, Manassas, Va.) have been shown to be susceptible to SARS CoV infection (Mossel, et al. (2008) Virology 372(1):127-135; Sainz, Jr., et al. (2004) Virology 329(1):11-17). Cells can be grown in Earle's minimal essential medium (Life Technologies, Inc.) supplemented with glutamine and fetal bovine serum. Studies have indicated that exposure of type II cells to hyperoxia leads to a rapid and reversible inhibition of cell proliferation. Such hyperoxic conditions include 5% CO₂, 95% O₂ atmosphere at 37° C. Under these hyperoxic conditions, cells cease proliferation after 24 hours (Clement, et al. (1992) J. Clin. Invest. 90:1812-1818; Corroyer, et al. (1996) J. Biol. Chem. 271:25117-25125). Likewise, treatment of Vero cells with 20 μM Lovastatin (Sigma) can arrest cells is G₁ (JavanMoghadam-Kamrani, et al. (2008) Cell Cycle 7:2434-40).

SARS-CoV strain Urbani can be obtained as a seed stock from Centers for Disease Control and Prevention, Atlanta, Ga. and propagated in Vero E6 cells. Vero E6 cells (American Type culture Collection, Manassas, Va.) are propagated in MEM supplemented with FBS. SARS-CoV titrations are performed on Vero E6 cells according to conventional methods (Sainz, Jr., et al. (2004) Virology 329(1):11-17).

What is claimed is:

1. A method for identifying an antiviral agent comprising
infecting a non-dividing host cell culture with an infectious virus that expresses a protease integral to the lifecycle of the virus;
contacting said host cell culture with a test agent and a peptide substrate for said protease;
incubating the host cell culture for a time sufficient to complete at least one lifecycle of the virus; and
determining activity of the protease using the peptide substrate, wherein a decrease of protease activity identifies the test agent as an antiviral agent.

2. The method of claim 1, wherein the virus is a Retroviridea virus, a Flaviviridea virus, a Picornaviridea virus, a Caliciviridea virus, a Togaviridea virus, or a Coronaviridea virus.

3. The method of claim 2, wherein the Flaviviridea virus is a hepatic virus.

4. The method of claim 1, wherein the host cell is permissive for viral infection and the culture is infected at a multiplicity of infection of less than 0.1 focus forming units/cell.

5. The method of claim 1, wherein the host cell culture is contacted with the test agent before or at the time of infection.

6. The method of claim 1, wherein the host cell culture is contacted with the test agent during the exponential phase of viral spread through the host cell culture.

7. The method of claim 1, wherein the lifecycle of the virus comprises host cell binding, entry, uncoating, translation, replication, assembly, maturation, egress and spread.

8. The method of claim 1, wherein the peptide is labeled.

9. The method of claim 8, wherein the label comprises dyes capable of fluorescence resonance energy transfer (FRET).

10. The method of claim 9, wherein FRET fluorescence is measured continuously, intermittently, or at a specified endpoint.

11. The method of claim 1, wherein the method is performed in a high-throughput manner.

12. The method of claim 1, further comprising the step of assessing the cytotoxicity of test agent.
